(19) United States
(12) Patent Application Publication
(10) Pub. No.: US 2018/0064968 A1
(43) Pub. Date: Mar. 8, 2018

(54) Wearable Air Purifier

(71) Applicant: Aleksandr Taslagyan, South Lake Tahoe, CA (US)

(72) Inventor: Aleksandr Taslagyan, South Lake Tahoe, CA (US)

(21) Appl. No.: 15/695,161

(22) Filed: Sep. 5, 2017

Related U.S. Application Data
(60) Provisional application No. 62/383,601, filed on Sep. 6, 2016.

(51) Int. Cl. A62B 23/06; B01D 46/44; B01D 46/24; B01D 46/00; A61L 9/20; A61L 9/22; A62B 23/02; A62B 18/08

(52) U.S. Cl. CPC A62B 23/06; B01D 46/44; B01D 46/2411; B01D 46/0032; A61L 9/20; A61L 2209/14; A62B 23/025; A62B 18/084; B01D 2275/10; A61L 2209/12; A61L 9/22

(57) ABSTRACT

Wearable Air Purifier consists of two arms behind the ears and the third support point in the area between the nostrils and the upper lip. It allows use of different types or combination of UV light sterilization, filter capturing particles suspended in the air and air ionization as well as capability of monitoring and notifying about air quality and/or sending the data to a personal electronic device such as a mobile phone. Air flow can occur naturally by the force of human lungs during inhalation and exhalation or forced air flow provided by air blowing device powered by a battery inside the enclosure to supply cleaned air into the space under the nostrils. Enclosure and arms forms, shapes, colors and materials can be a matter of fashion, depend on combination of air purification modules, or type of power source such as internal rechargeable battery or removable batteries. The whole structure of the device is made collapsible or foldable for ease of transportation and storage.

### BACKGROUND OF INVENTION

The present invention utilizes a method of wearing a personal air purification device.

It is an object of the invention to allow a convenient way of wearing the personal air purification device and easily foldable, storable design.

### SUMMARY OF THE INVENTION

The general purpose of the present invention which will be described subsequently in greater detail, is to provide a convenient way of wearing personal air purification device with integrated different types of air purification modules with or without forced air flow supply, ability to monitor air quality and a possibility of an attachable face mask covering the mouth.

To attain this, the present invention, Wearable Air Purifier form now on referred to as WAP, has two arms that are placed onto the ears and the third point of support is above the upper lip and under the nostrils, first two are on the ears. Instead of the two arms there can be a band going above the ears and around the head. It is to have various air purification and sterilization methods based modules built into it or their combination and sensors capable to monitor and notify about the air quality by audio or visual signals and or send the data wirelessly to a phone or other personal electronic device.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1a-c illustrates the method of wearing and several possible designs of the present invention.

FIG. 2a-b are views illustrating exterior features of WAP, passing the air through WAP and folding of the arms.

FIG. 3a-b are views illustrating several possible designs of the present invention.

FIG. 4a-b are views illustrating possible arrangements filters removal for replacement or cleaning.

FIG. 5a-b are cut-away views illustrating possible check valves arrangement inside the enclosure in case of not forced air flow.

FIG. 6a-d are views illustrating face mask attachment.

### DETAILED DESCRIPTION OF THE INVENTION

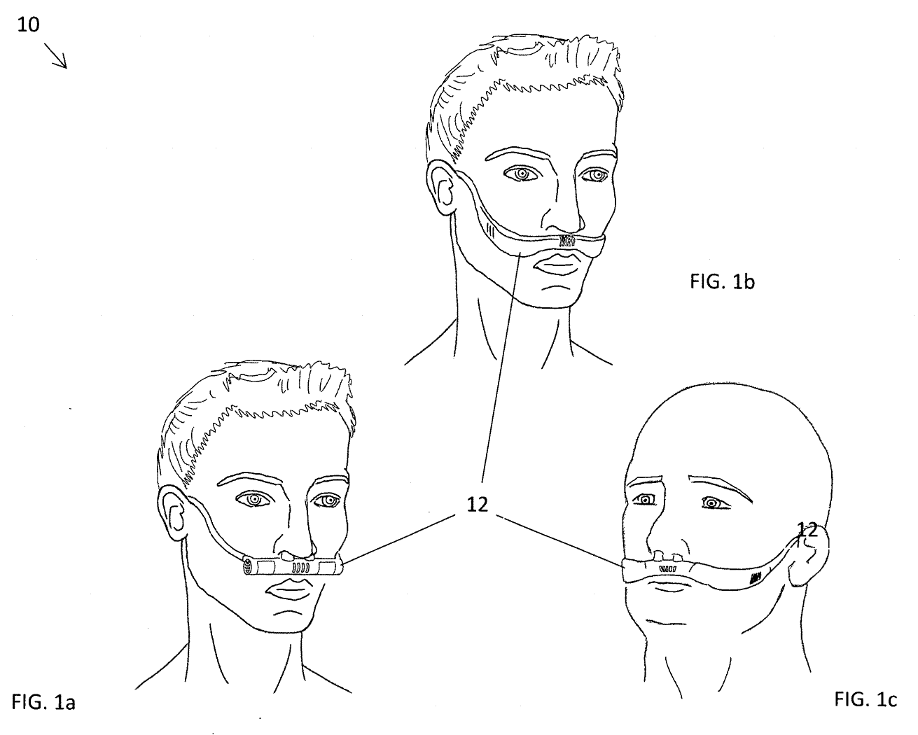

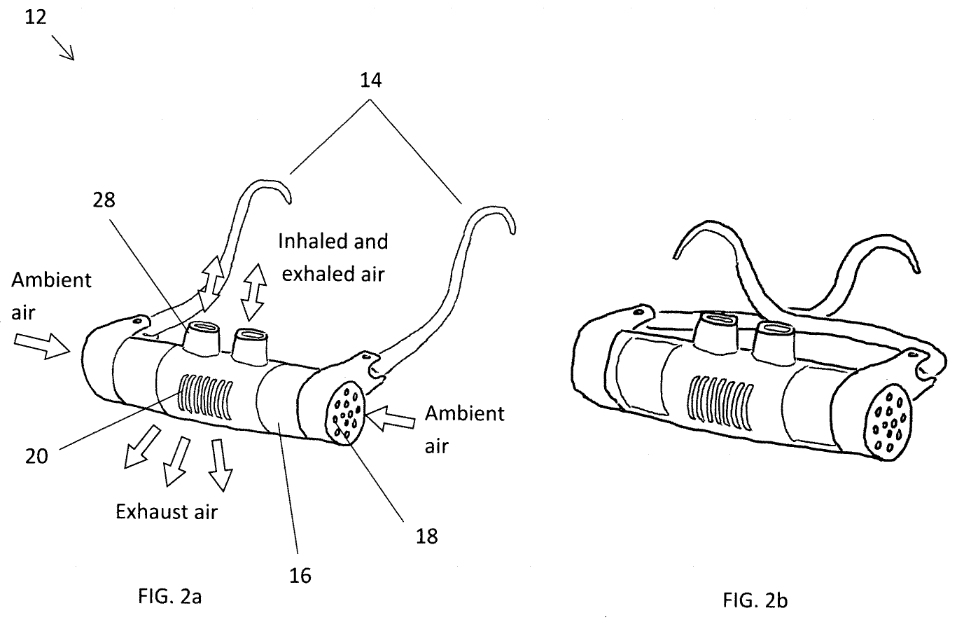

The idea being patented is the method of wearing 10 of the WAP 12 and WAP design allowing such method of wearing illustrated in FIG. 1a-c. WAP design utilizes two arms 14 which are hooked onto or placed on or around the ears and the third support point is in the area between the nostrils and the upper lip. Instead of two arms behind ears there can be a band going above the ears and around the head. Enclosure 16 and arms 14 forms, shapes, colors and materials can vary significantly. Whole structure of the device is collapsible or foldable for the ease of transportation and storage, see FIG. 2b. Hinged arms can be done collapsible around enclosure in a number of different ways.

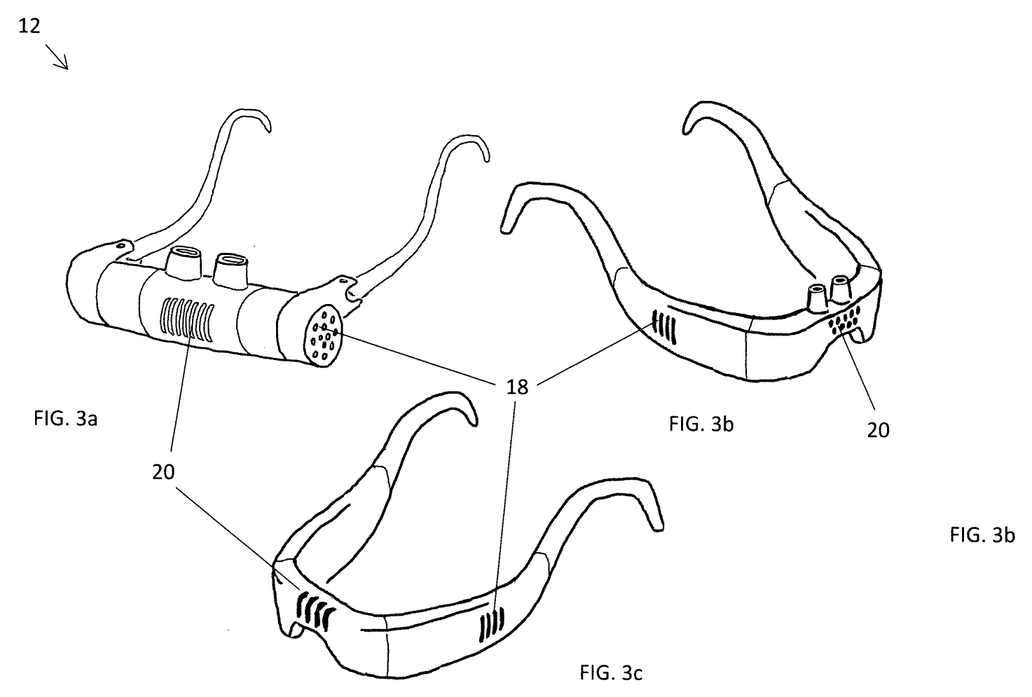

There are two methods providing the air flow through the device. First method is when air flow occurs naturally by the force of human lungs during inhalation and exhalation—not forced air flow. Second method is when WAP has an air blowing device which is powered by a battery inside the enclosure—forced air flow. FIG. 3a-c illustrates three design possibilities for WAP. FIG. 3a and FIG. 3b show designs that may either have forced or not forced air flow. FIG. 3c illustrates a design that can only have forced air flow as it lacks the inserts in nostrils and it utilises a miniature air blowing device inside the enclosure that draws air into WAP through air intake vents and supplies cleaned air into the space under the nostrils.

Ambient air is drawn into WAP through air inlet openings 18 and forced out of the device through air outlet openings 20, see FIG. 3a-c. Air intake openings 18 and outlet openings 20 can be of different shape or holes patterns and located in different areas of the enclosure 16. As air passes through the device it goes through either one or combination of air filters capturing the impurities suspended in the air, and/or UV light sanitization killing the pathogens suspended in the air, and/or air ionization devices imbedded inside WAP. Air purification modules, filters, batteries, wireless data transmitter are inside the enclosure. In case of several air cleaning modules they are nested one after the other along the airflow path.

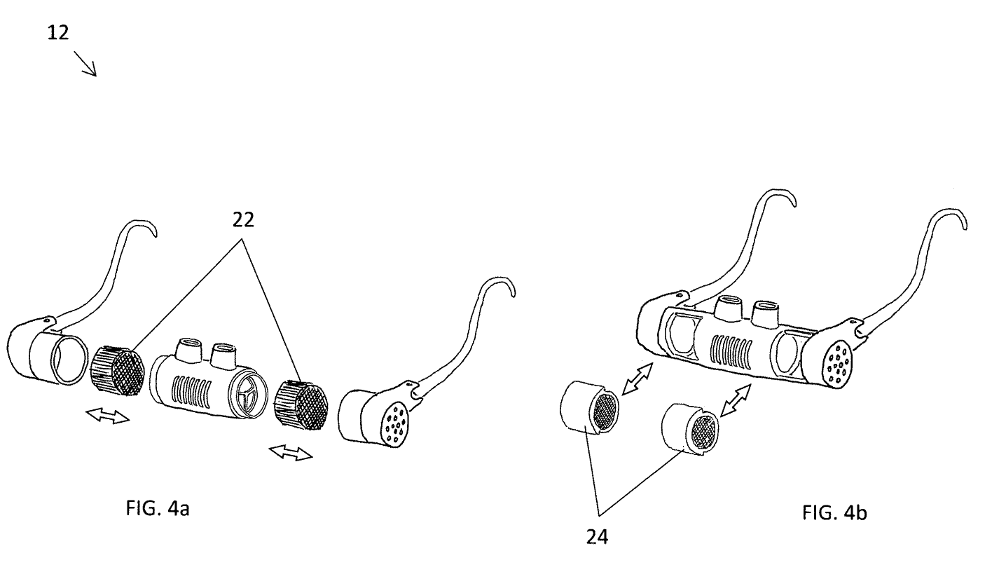

Filters can be made of several filtering layers and materials. Filters can be replaceable with the cartridge 24 they are positioned in or just the filtering material itself 22. They can also be either disposable or washable. The shape of the filters and the method how filters are installed in and removed from the enclosure may be utilized in many different ways. FIG. 4a illustrates removable filter material and FIG. 4b illustrates filter material in a cartridge configuration.

Air is passed in and out of the nostrils through a pair of tubular inserts 28 partially inserted into the nose of the person wearing the WAP. Inserts 28 can be detachable, washable and disposable.

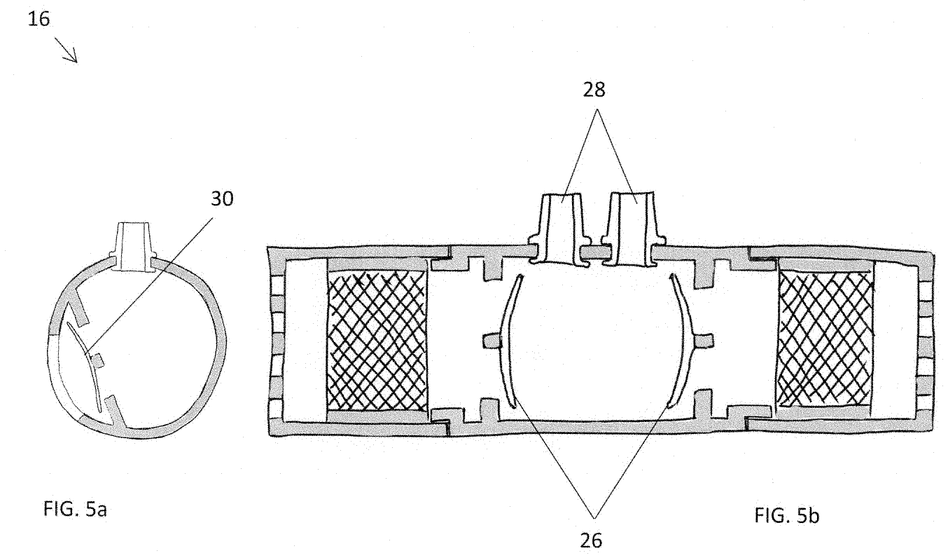

In case of not forced air flow there are air inlet check valves 26 allowing drawing ambient air through the WAP during inhalation illustrated in longitudinal cross-section view in FIG. 5b and air outlet check valve 30 allowing ejecting air through the exhaust vents during exhalation illustrated in transversal cross-section view in FIG. 5a. Illustrated in FIGS. 5a-b are flexible membrane type check valves but any type of check valves can be used.

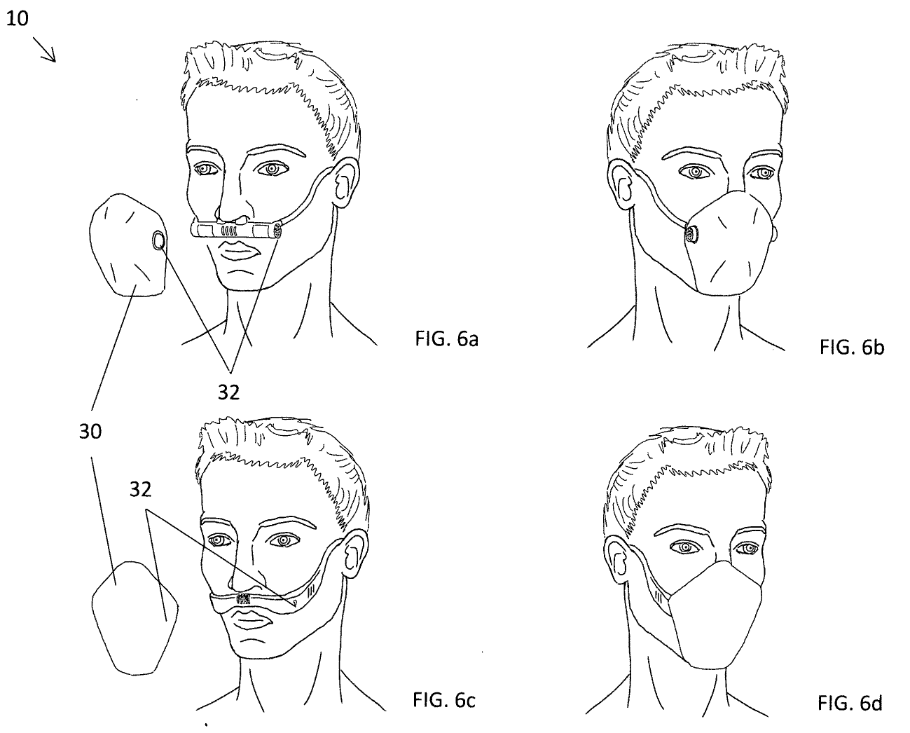

In case if there is a need to cover the mouth as well, there is also a possibility and means of attaching the face mask 30 to WAP with a quick attach and release attachment means 32 such as a pair of interlocking geometry and/or magnetic type connection spots on the face mask and on the WAP illustrated in FIG. 6a-b. The regular standard face masks can also be readily worn over the WAP to cover the mouth.

### INDEX OF ELEMENTS

10 Invention
12 Wearable air purifier
14 Arms
16 Enclosure
18 Air inlet openings
20 Air outlet openings
22 Filter
24 Filter cartridge
26 Air inlet check valve
28 Nostrils inserts
30 Air outlet check valve
32 Face mask attachment means
34
36
38
40
42
44
46
48
50
52
54
56
58
60
62
64
66
68
70
72
74
76
78
80
82
84
86
88
90
92

We claim:

1. The method of wearing the WAP, two arms behind the ears and the third support point is in the area between the nostrils and the upper lip.

2. The device as set forth in claim 1 utilizes air flow through the device. Ambient air enters air inlet openings and is passed through one or several air purification techniques based modules inside of the device, then cleaned air is directed towards the nostrils to be inhaled. As air passes through the device it goes through either one or combination of air filters capturing the impurities suspended in the air and/or UV light sanitization module section killing the pathogens suspended in the air by UV light, and/or air ionization module. Air purification modules, filters, batteries and wireless data transmitter are nested inside of the enclosure. In case of several air cleaning modules they are nested one after the other along the airflow path.

3. Air flow can occur naturally by the force of human lungs during inhalation and exhalation and a number of check valves arranged such that during inhalation they allow the ambient air to be drawn through air inlet openings then through air purification means inside the enclosure and towards nostrils. During exhalation check valves direct exhaled air out of outlet vents. Check valve or valves can also be arranged such that during exhalation the force required to open exhaust air outlet check valve is less that force to overcome resistance occurring during passing air through air purification means, such as air filter, and in this case check valve required only for opening and closing the exhaled air outlet passage.

4. Alternative to natural air flow option there can be a forced air flow provided by air blowing device powered by a battery inside the enclosure to supply cleaned air into the space under the nostrils.

5. Filters inside of the device can be made of several filtering layers and materials. Filters can be replaceable with the cartridge they are positioned in or just the filtering material itself. They can also be either disposable or washable. The shape of the filters and/or filter cartridges and the method how they are installed in and removed from the enclosure may be utilized in many different ways.

6. For natural, by the force of lungs, air flow option and as a possibility for powered air flow option air is passed in and out of the nostrils through a pair of inserts partially inserted into the nostrils of the person wearing the device. Nostrils inserts can be detachable, washable or disposable.

7. Device also allows for a possibility of attaching face mask to cover the mouth with a quick attach and release attachment means such as interlocking geometry or magnetic type connection spots located on the face mask and on the device. Standard face masks can also be readily worn over the device to cover the mouth.

8. The whole structure of the device can be made collapsible or foldable in a number of different ways for ease of transportation and storage.

9. Device has the ability to house air quality sensors for monitoring and notifying about air quality and/or sending the data to a personal electronic device such as a mobile phone.

10. Device enclosure and arms forms, shapes, colors and materials can be of different shapes, textures, colors and materials and be a matter of fashion and/or depend on combination of air purification modules, and/or types of power source such as an internal built-in rechargeable battery or removable batteries.
